(19) United States
(12) Patent Application Publication
(10) Pub. No.: US 2019/0290857 A1
(43) Pub. Date: Sep. 26, 2019

(54) PLUNGER ROD FOR UNDER-FILLED SYRINGES

(71) Applicant: FRESENIUS KABI USA, LLC, Lake Zurich, IL (US)

(72) Inventor: Anthony D. FERRARO, Lake Zurich, IL (US)

(21) Appl. No.: 15/933,711

(22) Filed: Mar. 23, 2018

(51) Int. Cl. A61M 5/315

(52) U.S. Cl. CPC A61M 5/31513; A61M 2005/31523; A61M 5/31515

(57) ABSTRACT

A plunger rod for use with a syringe barrel includes a first flange adjacent a distal end of the plunger rod, a second flange adjacent a proximal end of the plunger rod opposite the distal end, and a securing flange between the first flange and the second flange. The securing flange is configured to prevent movement of the plunger rod in a proximal direction beyond a predetermined position when the plunger rod is disposed within the syringe barrel. A syringe assembly and a method of preparing a syringe assembly are also disclosed.

### TECHNICAL FIELD

This disclosure generally relates to devices and methods of using syringes containing medical materials, and more particularly relates to a new design for a plunger rod for use with under-filled syringes.

### BACKGROUND

Syringe assemblies are used to hold, transport, and deliver materials. For example, syringes are often utilized in medical environments to administer one or more medicinal components. Syringe assemblies may differ in size, and the specific dimensions are dictated by the desired application and the specific material to be administered.

Syringes generally are provided in two forms: empty or prefilled. Empty syringes are intended to be filled by an end user from a vial or other container, whereas prefilled syringes are supplied to an end user with a liquid, e.g., medicament or diluent, provided therein by a manufacturer. In certain instances, syringes are provided with means to prevent the stopper from inadvertently pulling out of the syringe barrel, such as a backstop device.

In some instances, syringes may be pre-filled with one or more materials that are then dispensed from the syringe and combined with other elements (e.g., medicament, solvent, reactive components, etc.) to form a mixture. This mixture can then be moved back into the syringe assembly for immediate or future administering.

There are shortcomings with existing technology for reconstitution of materials within a syringe assembly. In some existing environments, when the reconstituted mixture is introduced into the syringe assembly, a portion of the mixture enters a part of the assembly that may not be sterile. This can lead to contamination.

Therefore, there is a need for improved syringe assemblies and methods of reconstituting materials.

### SUMMARY

The foregoing needs are met by the various embodiments of syringe assemblies and syringe plungers disclosed. According to an aspect of the present disclosure, a plunger rod for use with a syringe barrel includes a first flange adjacent a distal end of the plunger rod, a second flange adjacent a proximal end of the plunger rod opposite the distal end, and a securing flange between the first flange and the second flange. The securing flange is configured to prevent movement of the plunger rod in a proximal direction beyond a predetermined position when the plunger rod is disposed within the syringe barrel.

According to another aspect, a syringe assembly includes a syringe barrel and a plunger rod. The syringe barrel has an interior surface defining a chamber that is configured to receive a material. The plunger rod is configured to slidably move within the chamber along a dispensing axis extending from a distal end to a proximal end of the syringe barrel. The plunger rod has a plunger rod body with a distal end and a proximal end opposite the distal end, a first flange adjacent the distal end, a second flange adjacent the proximal end, and a securing flange between the first flange and the second flange. The syringe assembly further includes a backstop adjacent the proximal end of the syringe barrel. The backstop is configured to contact the securing flange to prevent the securing flange from passing through the backstop.

According to another aspect, a method of preparing a syringe assembly for administering a medicament includes receiving a first medicament component within a syringe barrel of a syringe, moving a plunger rod in a distal direction to dispense the first medicament component into contact with a second medicament component such that a mixture is formed comprising the first and second medicament components, and moving the plunger rod in a proximal direction opposite the distal direction to draw the mixture into a sterile portion of the syringe barrel. The movement of the plunger rod in the proximal direction is limited by contact of a securing flange extending from the plunger rod and a backstop on the syringe.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates an isometric view of a syringe assembly according to an aspect of the present disclosure;

FIG. 2 illustrates an isometric view of a plunger rod according to an aspect of the disclosure;

FIG. 3 illustrates an isometric view of a syringe barrel according to an aspect of the disclosure;

FIG. 4 illustrates a side perspective view of a syringe assembly having a plunger rod at a predetermined maximum distance from the distal end of the barrel according to an aspect of the disclosure;

FIG. 5 illustrates a side perspective view of the syringe assembly of FIG. 4 having the plunger rod disposed adjacent the distal end of the barrel;

FIG. 6 illustrates an isometric view of a backstop according to an aspect of the disclosure; and

FIG. 7 illustrates a front perspective view of the backstop of FIG. 6.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

Disclosed are aspects of devices and methods for operating a syringe assembly with a plunger rod that prevents movement of the material within the syringe beyond a predefined point in the syringe. In some aspects, a syringe assembly 10 includes a plunger rod 100 disposed within a syringe barrel 200.

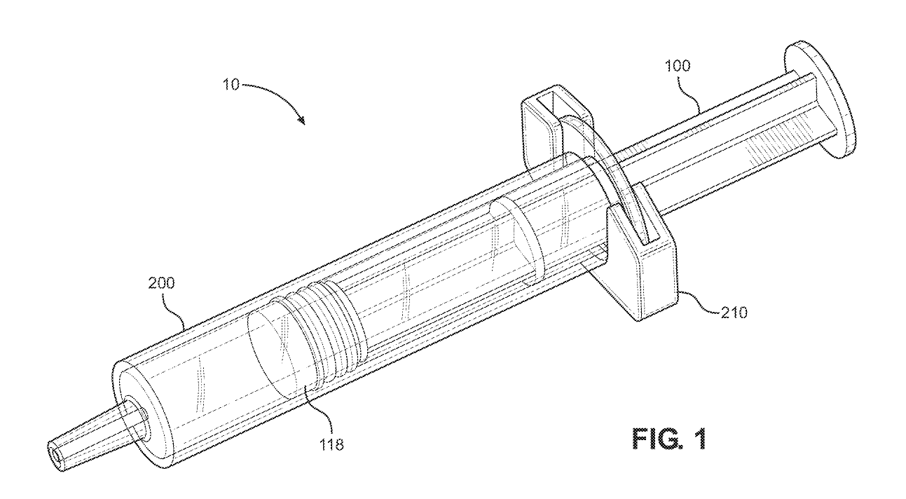

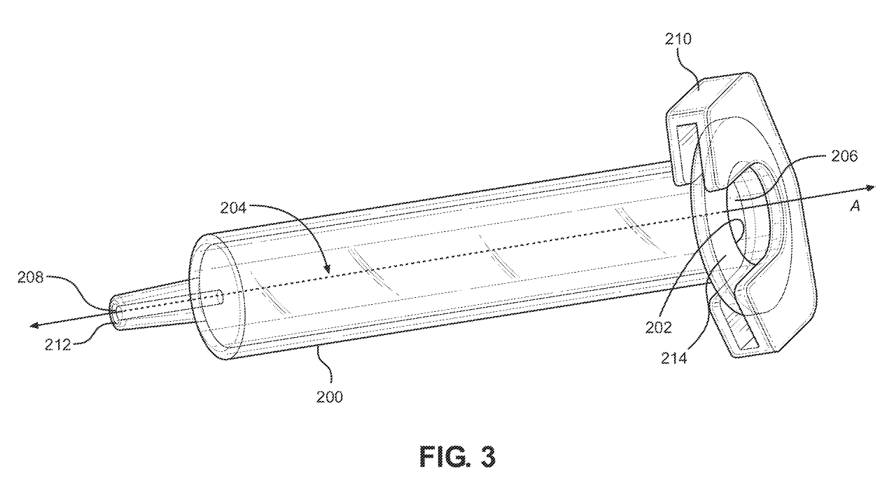

Referring to FIGS. 1-5, the syringe barrel 200 may include a substantially cylindrical hollow portion having an interior surface 202 that defines a chamber 204. The chamber 204 is configured to receive one or more materials for storing, mixing, and/or dispensing. The plunger rod 100 may be disposed within the chamber 204 and may be configured for slidable movement therein.

The syringe barrel 200 includes a distal end 212 at one end of the syringe barrel 200 and a proximal end 214 opposite the distal end 212. In some aspects, the distal end 212 may include a connection interface 208 for engagement with an external tool (not shown), for example, a syringe needle, a medical transport line, or a container. The connection interface 208 may be tapered and/or a Luer connection.

At the proximal end 214, the syringe barrel 200 includes an opening 206 in fluid communication with the chamber 204. The plunger rod 100 may pass through the opening 206, with at least a portion of the plunger rod 100 being movable between the inside of the chamber 204 and the outside of the chamber 204.

A backstop 210 is disposed adjacent the proximal end 214 to prevent the plunger rod 100 from moving beyond a predetermined threshold into or out of the chamber 204. The backstop 210 may contact one or more flanges on the plunger rod 100 as described below in detail. The backstop 210 is dimensioned such that a portion of the plunger rod 100, for instance the plunger rod body 102 proximal of a flange, may pass through, while another portion of the plunger rod body 102 distal of the flange is prevented from moving past the backstop 210.

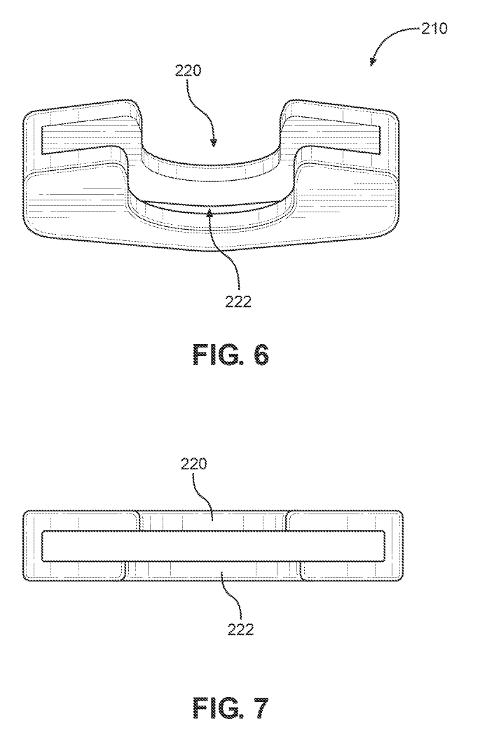

The backstop 210 may be integral with the syringe barrel 200 such that the syringe barrel 200 and the backstop 210 form one unitary component. Alternatively, the backstop 210 may be releasably connected to the syringe barrel 200. For example, the backstop 210 may be attached to the syringe barrel 200 after the plunger rod 100 is inserted into the chamber 204. The backstop 210 may include one or more openings through at least a portion of the plunger rod 100 may pass. For example, referring to FIGS. 6 and 7, the backstop 210 may include a proximal opening 220 and a distal opening 222. The proximal and distal openings 220, 222 may have different dimensions and shapes. The proximal opening 220 may be dimensioned such that the effective size of the opening 206 is reduced when the backstop 210 is attached to the proximal end 214 of the syringe barrel 200.

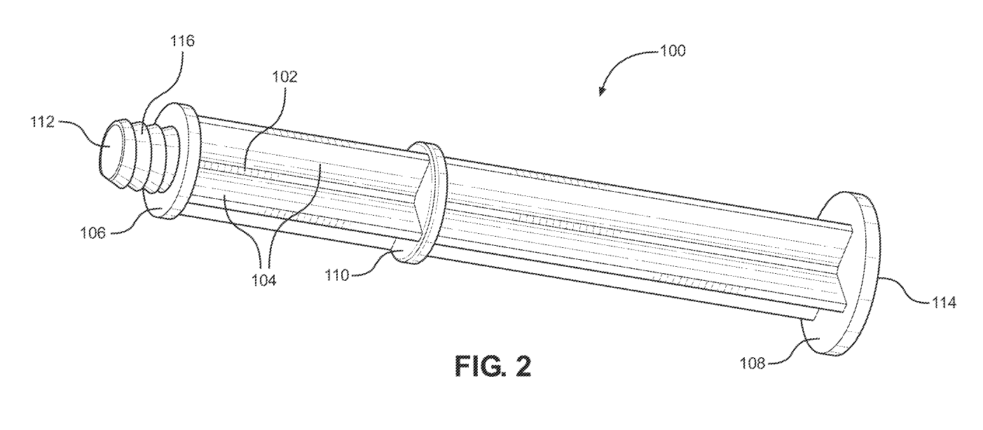

Referring still to FIGS. 1-5, the plunger rod 100 includes a plunger rod body 102 having a distal end 112 at one side and a proximal end 114 at the opposite side thereof. The plunger rod body 102 may be any suitable shape, such as cylindrical or prismatic, and may have one or more support structures for maintaining rigidity of the plunger rod body 102. In some aspects, one or more walls 104 may extend radially from the plunger rod body 102 along at least a portion of the plunger rod body 102. Referring to the exemplary illustrative aspect of FIG. 2, the plunger rod 100 may have four radially extending walls 104 forming a substantially “X”-shaped cross section of the plunger rod body 102. It will be understood that other cross sections are possible, and the number of extending walls 104 may differ, for example, 1, 2, . . . , or 8 walls 104. In some aspects, the plunger rod 100 may be devoid of additional support structures or walls extending therefrom resulting in a round cross section.

The distal end 112 of the plunger rod 100 may include a connection interface 116 configured to receive a sealing member 118. The sealing member 118 may be a stopper, plunger, piston, plug, a gasket, a washer, or another suitable component. The sealing member 118 is sized and dimensioned such that it may move within the chamber 204 while slidably contacting the interior surface 202 of the syringe barrel 200. Referring again to FIGS. 1-5, the sealing member 118 may be a stopper that includes a compressible material that, when inside the chamber 204, prevents liquids from moving past it.

A first flange 106 may be disposed adjacent to the distal end 112 on the plunger rod 100. The first flange 106 may be extend radially from the plunger rod body 102 such that it provides a contact surface for the sealing member 118 disposed at the distal end 112. While it will be appreciated that the first flange 106 may have various shapes depending on desired materials and manufacturing processes, in some aspects disclosed herein, the first flange 106 has a substantially round cross section. In some aspects, the sealing member 118 may be a gasket or a sealing ring (such as an O-ring) that contacts the first flange 106 at the outermost portion of the first flange 106 closest to the interior surface 202 of the syringe barrel 200. It will be understood that the shape of the first flange 106 may be different, for example having one or more rectangular, triangular, or elliptical projections extending radially from the plunger rod body 102, and that the present disclosure is not limited only to the disclosed circular shape. In addition, in some aspects, the first flange 106 may include multiple adjacent flanges to provide additional stability when inserting the plunger rod 100 in the chamber 204.

The first flange 106 may be sized such that its outer diameter is smaller than the inner diameter of the syringe barrel 200 to permit movement of the plunger rod 100 within the syringe barrel 200 along a dispensing axis A. In some aspects, the first flange 106 may be sized such that it cannot move past the backstop 210. Such a design prevents the plunger rod 100 from being removed from the syringe barrel 200 and maintains at least a portion of the plunger rod 100 within the chamber 204.

The plunger rod 100 may include a second flange 108 adjacent the proximal end 114. The second flange 108 may function as an interface for moving the plunger rod 100 distally and proximally within the syringe barrel 200. The second flange 108 may have the same shape as the first flange, or it may have a different suitable shape. The outer diameter of the second flange 108 may be smaller than, larger than, or the same size as the outer diameter of the first flange 106. The second flange 108 may be contacted by a user's thumb, finger, palm, or an external tool to slidably move the plunger rod 100 within the syringe barrel 200. Because of this, it will be understood that the second flange 108 is not limited to a particular shape or dimensions exemplified throughout this disclosure, and that another suitable shape for the second flange 108 may be used.

With continued reference to FIGS. 1-5, the second flange 108 as shown is substantially circular and has a larger outer diameter than the first flange 106. The second flange 108 may be shaped and dimensioned such that it cannot pass through the opening 206 in the syringe barrel 200 and/or move past the backstop 210.

The plunger rod 100 may further include a securing flange 110 fixedly attached to the plunger rod body 102 between the distal end 112 and the proximal end 114. The securing flange 110 may be dimensioned and shaped similarly to the first flange 106 or the second flange 108, or it may have different dimensions. The securing flange 110 has an outer diameter that is smaller than the inner diameter of the syringe barrel 200. Additionally, the securing flange 110 may be shaped and sized such that it cannot pass through the opening 206 when the backstop 210 is attached to the proximal end 214 of the syringe barrel 200. It will be appreciated that the outer diameter of the securing flange 110 may be greater than the outer diameter of the first flange 106 and/or the second flange 108, smaller than the outer diameter of the first flange 106 and/or the second flange 108, or the same as the outer diameter of the first flange 106 and/or the second flange 108. Referring to the illustrative aspects of FIGS. 1-5, the securing flange 110 has an outer diameter that is greater than that of the first flange 106, but smaller than that of the second flange 108.

While the present disclosure describes a substantially cylindrical barrel and circular flanges that correspond to the circular cross section of the barrel, it will be understood that other shapes of the syringe barrel 200 are possible, such as elliptical or polygonal, and that the flanges and other components of the syringe assembly 10 can have correspondingly different shapes as well to conform to the shape of the syringe barrel 200.

It may be advantageous to prevent the plunger rod 100 from moving beyond a predetermined distance away from the distal end 212. To prevent the plunger rod 100 from moving past the predetermined distance, the securing flange 110, which is fixedly attached to the plunger rod 100, may contact the backstop 210. The portion of the plunger rod body 102 proximal of the securing flange 110 may be configured to pass through the backstop 210, while the portion of the plunger rod body 102 distal of the securing flange 110 may be precluded from passing through the backstop 210.

As described above, some aspects of the backstop 210 may include a plurality of openings. Referring again to FIGS. 6 and 7, the diameter of the proximal opening 220 may be smaller than the diameter of the distal opening 222. In some aspects, the securing flange 110 may have a diameter that is greater than that of the proximal opening 220 and smaller than the diameter of the distal opening 222. In such a configuration, the securing flange 110 can pass through the distal opening 222 but not through the proximal opening 220. The securing flange 110, having a larger diameter than the proximal opening 220, would contact the backstop 210 adjacent the proximal opening 220 and would not be able to pass through.

The particular axial position of the securing flange 110 on the plunger rod 100 governs the desired predetermined maximum distance that the plunger rod 100 may travel away from the distal end 212 of the syringe barrel 200. When the plunger rod 100 is at the predetermined maximum distance, the portion of the chamber 204 defined by the interior surface 202 of the barrel, the distal end 212, and the distal end 112 of the plunger rod has a predetermined maximum volume.

The predetermined maximum volume may be modified by selecting a differently sized syringe barrel 200, by changing the size and shape of the distal end 112 of the plunger rod 100 (and/or of the sealing member 118), and/or by changing the placement of the securing flange 110 along the plunger rod body 102. In such aspects, the closer the securing flange 110 is to the distal end 112, the greater the predetermined maximum distance and the subsequent predetermined maximum volume will be; the closer the securing flange 110 is to the proximal end 114, the smaller the predetermined maximum distance and the subsequent predetermined maximum volume will be.

The plunger rod 100 as described in this disclosure may be used with existing syringe barrels 200. While syringe barrels exist in various shapes, sizes, and capacities, commonly available syringe barrels often have volumes of 1 mL, 3 mL, 5 mL, or 10 mL. In some aspects, the desired quantity of fluid introduced into a syringe assembly is not the same as one of the standard existing syringe barrel volumes. In such cases, the syringe assembly may be under-filled, where the volume of fluid inside the syringe barrel is less than the total capacity of that barrel.

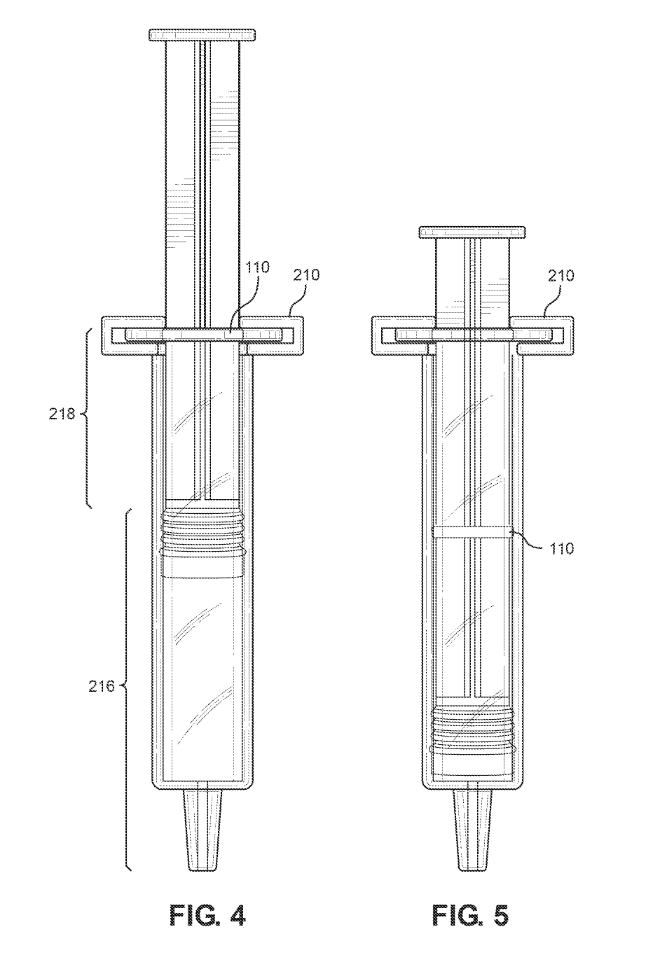

In some aspects, the syringe assembly may be pre-filled with a desired material, for example a medicament, at a different time or location from use. A portion of the syringe assembly may be sterilized or maintained in a sterile environment. Referring to FIGS. 4 and 5, the syringe assembly may have a sterile portion 216 where the fluid is located between the distal end 212 of the syringe barrel 200 and the sealing member 118 of the plunger rod 100. A non-sterile portion 218 may exist within the syringe barrel 200 adjacent to the sterile portion 216. It will be understood that the terms “sterile” and “non-sterile” are relative terms and refer to the sterility of a component as it compares to another component. While the entire syringe assembly 10 may have been sterilized at one point, the non-sterile portion 218 may have lost the sterility during transportation or use of the syringe assembly 10. This may be due to the opening 206 not being sealed in an air-tight manner, for example.

The pre-filled syringe may include a fluid that is to be mixed with another component to form a mixture, and that mixture may then be introduced into the syringe barrel 200. A first component within the pre-filled syringe may be dispensed by moving the plunger rod 100 in a distal direction toward the distal end 212 of the syringe barrel 200 and combined with a second component to form a mixture. When the first and second components are combined, the mixture may be moved into the syringe by moving the plunger rod 100 in a proximal direction opposite the distal direction and toward the proximal end 214 of the syringe barrel 200. The mixture may then be dispensed from the syringe assembly by again moving the plunger rod 100 in the distal direction.

When the mixture is formed and moved into the syringe barrel 200, it may be advantageous to prevent any of the mixture from contacting or occupying any of the non-sterile portion 218 in order to keep the mixture uncontaminated.

In some aspects, the first component is a liquid, which may be aqueous, non-aqueous, or a combination of aqueous and non-aqueous liquids. Exemplary first components include, but are not limited to water, 0.9% saline, 5% dextrose, Ringer's lactate solution, and other pharmaceutically acceptable diluents. In other aspects, the first component may be a liquid composition comprising a medicament and, optionally, other pharmaceutically acceptable excipients. The second component may be a solid or a liquid. In some aspects, the second component may be a solid, such as powdered medicament. In other aspects, the second component may be a liquid, such as a liquid composition comprising a medicament and, optionally, other pharmaceutically acceptable excipients. When the first and second components are combined, the mixture can take on any suitable physical form including, but not limited to, solution, suspension, emulsion, or dispersion.

The predetermined distance that the plunger rod 100 is allowed to travel may correspond to the farthest the plunger rod 100 may travel away from the distal end 212 of the syringe barrel 200 before the mixture within the sterile portion 216 begins to contact the non-sterile portion 218. The predetermined maximum volume may also correspond with the necessary volume of the mixture while prohibiting excess mixture volume from entering the syringe barrel 200 and moving into the non-sterile portion 218.

As described above, the securing flange 110 may be disposed on the plunger rod 100 to contact the backstop 210 of the syringe barrel 200 to prevent the plunger rod 100 from moving farther than the predetermined maximum distance.

By preventing the user from inadvertently moving the plunger rod 100 too far away from the distal end 212, the risk of contaminating the material decreases. Additionally, user error is lowered because the user does not need to actively monitor how far the plunger rod 100 has moved. This decreases work-related stress for the user and allows for faster, more accurate, and more consistent treatment.

The predetermined maximum distance that the plunger rod 100 is permitted to travel and the related predetermined maximum volume of the chamber that defines the sterile portion 216 may additionally prevent the material in a pre-filled syringe from moving into the non-sterile portion 218. During storage or transportation of pre-filled syringes, environmental pressure and/or temperature changes can cause the pre-filled volume to expand or contract. The pre-filled fluid may expand in volume and push against the sealing member 118 or the first flange 106, resulting in movement of the plunger rod 100 away from the distal end 212 of the barrel. This may permit the fluid to contact the non-sterile portion 218 of the barrel. Providing the securing flange 110 that prevents the plunger rod 100 from moving beyond the predetermined maximum distance, the pre-filled fluid may be precluded from entering any part of the non-sterile portion 218.

It will be understood that the predetermined maximum volume can be varied based on the desired volume of the material in the sterile portion 216 of the chamber 204. The predetermined maximum volume may be dependent on the placement of the securing flange 110 along the plunger rod 100 and the related predetermined maximum distance that the plunger rod 100 is permitted to travel away from the distal end 212 of the syringe barrel 200.

The desired volume may be calculated in any suitable way, such as by adding fluid into the chamber until the desired space is filled and then measuring the amount of fluid that was used to fill the space. In some aspects, additional volume may be added to the measured amount to allow for a desired amount of air or other gas. Based on the measured volume, the securing flange 110 may be positioned along the plunger rod body 102 such that the plunger rod 100 is precluded from moving beyond the predetermined maximum distance where the volume of the chamber would surpass the measured predetermined maximum volume.

The plunger rod 100 having the securing flange 110 may be manufactured via any suitable means, for example, by casting the plunger rod 100 with the securing flange 110 in a mold. The mold may be adjusted to produce plungers having one or more flanges along different portions of the plunger rod body 102. Different molds are possible to accommodate various sizes of syringe assemblies and different positioning of the flanges.

While the illustrative aspects depict the syringe assembly 10 as described in detail throughout this disclosure, it will be appreciated that various modifications are possible for the plunger rod 100 and/or the syringe barrel 200. In some aspects, the plunger rod 100 may include additional flanges (not shown) disposed along the plunger rod body 102 that may, for example, increase rigidity of the plunger rod 100.

A pre-filled syringe assembly 10 may be assembled by introducing one or more components, for example medical components, into the syringe barrel 200. To secure the components, the plunger rod 100 may be introduced into the syringe barrel 200. In some aspects, a backstop 210 may be disposed adjacent the proximal end 214 after the plunger rod 100 is introduced into the syringe barrel 200. In some aspects, it may be advantageous for the plunger rod 100 to include one or more sealing members 118 to improve the decrease the risk of leakage from within the syringe barrel 200. The sealing member 118 may be integral with the plunger rod 100, or it may be affixed to the plunger rod 100 before introducing the plunger rod 100 into the syringe barrel 200. The sealing member 118 may be attached to the plunger rod 100 via a threaded connection, an interference fit, an adhesive, or another suitable attachment method. After the plunger rod 100 is positioned in the syringe barrel 200 at the desired location, a lock (not shown) may be disposed on the syringe barrel 200, the plunger rod 100, or both to prevent the plunger rod 100 from prematurely moving within the syringe barrel 200 during storage or transportation.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

What is claimed is:

1. A plunger rod for use with a syringe barrel, the plunger rod comprising:
a plunger rod body having a distal end and a proximal end opposite the distal end;
a first flange adjacent the distal end;
a second flange adjacent the proximal end; and
a securing flange between the first flange and the second flange,
wherein the securing flange is configured to prevent movement of the plunger rod in a proximal direction beyond a predetermined position when the plunger rod is disposed within the syringe barrel.

2. The plunger rod of claim 1, wherein the outer diameter of the securing flange is greater than the outer diameter of the first flange.

3. The plunger rod of claim 2, wherein the outer diameter of the second flange is greater than the outer diameter of the first flange and of the securing flange.

4. The plunger rod of claim 1, further comprising a plurality of walls extending radially from the plunger rod body.

5. The plunger rod of claim 1, further comprising a connection interface adjacent the distal end, the connection interface being configured to receive a sealing member.

6. The plunger rod of claim 1, wherein a portion of the plunger rod body proximal of the securing flange is configured to pass through a backstop of the syringe barrel, and a portion of the plunger rod body distal of the securing flange is precluded from passing through a backstop of the syringe barrel.

7. A syringe assembly, comprising:
a syringe barrel having an interior surface defining a chamber, the chamber configured to receive a material;
a plunger rod configured to slidably move within the chamber along a dispensing axis extending from a distal end of the syringe barrel to a proximal end of the syringe barrel, the plunger rod having:
a plunger rod body having a distal end and a proximal end opposite the distal end;
a first flange adjacent the distal end;
a second flange adjacent the proximal end; and
a securing flange between the first flange and the second flange; and
a backstop adjacent the proximal end of the syringe barrel, the backstop configured to contact the securing flange to prevent the securing flange from passing through the backstop.

8. The syringe assembly of claim 7, wherein the outer diameter of the securing flange is greater than the outer diameter of the first flange.

9. The syringe assembly of claim 8, wherein the outer diameter of the second flange is greater than the outer diameter of the first flange and of the securing flange.

10. The syringe assembly of claim 7, further comprising a sealing member, wherein the plunger rod body comprises a connection interface adjacent the distal end of the plunger rod body, the connection interface being configured to receive the sealing member.

11. The syringe assembly of claim 7, wherein a portion of the plunger rod body proximal of the securing flange is configured to pass through the backstop, and a portion of the plunger rod body distal of the securing flange is precluded from passing through the backstop.

12. The syringe assembly of claim 7, wherein the backstop is releasably connected to the syringe barrel.

13. The syringe assembly of claim 12, wherein the backstop defines a proximal opening and a distal opening, the proximal opening being smaller than the distal opening.

14. The syringe assembly of claim 13, wherein the proximal opening has a diameter smaller than the diameter of the securing flange.

15. A method of preparing a syringe assembly for administering a medicament, the method comprising:
receiving a first medicament component within a syringe barrel of a syringe;
moving a plunger rod in a distal direction to dispense the first medicament component into contact with a second medicament component such that a mixture is formed comprising the first and second medicament components; and
moving the plunger rod in a proximal direction opposite the distal direction to draw the mixture into a sterile portion of the syringe barrel, wherein the movement of the plunger rod in the proximal direction is limited by contact of a securing flange extending from the plunger rod and a backstop on the syringe.

16. The method of claim 15, wherein movement of the plunger rod in the proximal direction is limited such that the mixture does not move into a non-sterile portion adjacent the sterile portion.

17. The method of claim 15, wherein the outer diameter of the securing flange is greater than an opening in the backstop to limit the movement of the plunger rod in the proximal direction.

18. The method of claim 15, wherein the plunger rod further includes a connection interface configured to receive a sealing member, the method further comprising screwing the plunger rod to the sealing member after receiving the first medicament component within the syringe barrel of the syringe.

19. The method of claim 15, further comprising administering the mixture by again moving the plunger rod in the distal direction to dispense the mixture.

20. The method of claim 15, wherein the plunger rod comprises a first flange adjacent its distal end and a second flange adjacent its proximal end, the securing flange being between the first flange and the second flange.
